(19) United States
(12) Patent Application Publication
(10) Pub. No.: US 2024/0017086 A1
(43) Pub. Date: Jan. 18, 2024

(54) ILLUMINATION DEVICES, SYSTEMS AND METHODS

(71) Applicant: SYMBIOX, INC., San Diego, CA (US)

(72) Inventors: Jose-Tomás EGAÑA-ERAZO, Santiago (CL); Christian Dani GUZMÁN, Santiago (CL)

(21) Appl. No.: 18/033,523

(22) PCT Filed: Oct. 28, 2021
(86) PCT No.: PCT/US2021/057141

§ 371 Date: Apr. 24, 2023

Related U.S. Application Data
(60) Provisional application No. 63/107,375, filed on Oct. 29, 2020.

(51) Int. Cl. A61N 5/06; A61L 27/36; A61L 27/54; A61L 31/02

(52) U.S. Cl. CPC A61N 5/062; A61N 5/0601; A61L 27/3637; A61L 27/54; A61L 31/022; A61L 2300/10; A61N 2005/0652; A61N 2005/063; A61N 2005/0626; A61N 2005/0633

(57) ABSTRACT

Illumination devices, and illumination systems comprising illumination devices are provided herein. The illumination device can comprise a metal or other substrate, a living hinge, and a set of lighting elements supported by a set of printed circuit boards and connected via a set of cables. The set of lighting elements can be arranged in a rhomboid or hexagonal configuration. The illumination devices can be, for example, wearable, formed as a container, used with a container, or used inside of a body to illuminate a photosynthetic implant or other object.

### FIELD OF THE INVENTION

The field of the invention is illumination devices, systems and methods, especially those for use with living tissue, and compositions and devices used to treat living tissue.

### BACKGROUND

Lighting devices are useful in several applications in the fields of science and medicine. For example, red LED lights are often used for skincare treatments, for example, to treat acne, reduce inflammation, or promote anti-aging effects. Further, lights can be used to stimulate Chlorophyll A and B in photosynthetic scaffolds used to address issues of insufficient oxygen supply. U.S. Patent application publication no. 2016/0058861 to Symbiox Inc. teaches that photosynthetic cells, such as algal cells, can be used in these substances to provide the ability to continuously generate oxygen when exposed to a light source or other oxygen-generating trigger. Unfortunately, there are often blocking elements that prevent the cells from accessing the light required or helpful to generate oxygen. In an effort to address these concerns, the '861 Application also teaches that a light source can be embedded in a bandage to ensure that the cells continue to produce oxygen if the bandage otherwise blocks ambient light, and that a container housing an organ covered or filled with photosynthetic cells can be devised with a light source to help maintain the organ until it can be transplanted.

Unfortunately, many lighting devices are prone to overheating, which can be dangerous and lead to burns when used for long periods of time in close proximity to an organ or other parts of a person or animal. Additionally, it can be uncomfortable and difficult for a patient or other user to stand or sit near a lighting apparatus for long periods of time with restricted or little movement.

### SUMMARY

The present disclosure is directed toward one or more improved features identified below, and to methods that address the above-mentioned problems.

The inventive subject matter provides an illumination system comprising an illumination device. The illumination device can comprise a metal or other substrate, and a set of lighting elements coupled to the substrate. In some aspects, the substrate is preferably a copper substrate, but other substrates, including all suitable metals that are malleable and conductors of heat and electricity are contemplated. The substrate can comprise a living hinge comprising, for example, thinned or cut/cutout portions or any suitable size and shape to allow the more rigid metal sheet or other substrate to more easily bend (e.g., curve) along the lines of the hinge. In some embodiments, it is contemplated that one or more hinges may be provided wherein each hinge is a separate piece that movably couples a piece of the metal substrate to another piece. While some examples include a metal substrate, it should be appreciated that a substrate of any suitable material may be used (e.g., PDMS (silicon), a transparent acrylic).

The set of lighting elements can comprise any suitable light elements, including for example, light emitting diodes, light emitting portions of optical fibers, or a combination thereof. Fiber optics or optical fibers can be beneficial, for example, where heat is a concern. Since the light source can be separate from the point of illumination, the fiber can transmit the light and isolate the heat from the light source from the point of illumination. Further, fiber optics can be preferred as they can be nonconductive, can focus light precisely on an area, can be flexible, and may not require separate electrical cables. Light emitting diodes can be beneficial, for example, as a user can vary the resistances and the light intensity. Further, different color temperature LEDs can be used, and LEDs are generally more energy-efficient. Light emitting diodes can be supported by a set of printed circuit boards coupled to the metal or other substrate and connected via a set of cables. As used herein, a “lighting element” can comprise a single illumination point or multiple illumination points bundled or placed closely together (e.g., such that the distance between two illumination points of a single lighting element is no greater than 10% of the distance between the lighting element and another adjacent lighting element). In some aspects, the light source or set of lighting elements emit light at photosynthetically active wavelengths. In some aspects, the light source or set of lighting elements emit light at wavelengths of between 400-700 nm.

The illumination device can be wearable and can be formed to fit on any portion of a wearer's body. For example, the device can be formed as a face mask, a helmet, an arm band (e.g., having a cross-section with a diameter of between 3-12 inches or between 3-9 inches), a sleeve, a leg band (e.g., having a cross-section with a diameter of between 4-15 inches or between 4-12 inches), shorts, pants, a shirt, a belt, or a portion or combination thereof. In some embodiments, the device can comprise a band that includes an elastic element or strip that stretches to accommodate body parts of different sizes. In some aspects, it is contemplated that at least 80% or at least 90% of the band can comprise the metal or other substrate, and between 1-20% or between 1-10% of the band can comprise the elastic element. Additionally or alternatively, the wearable device can comprise suitable fasteners (e.g., straps, straps with hook or loop elements, hook fasteners, loop fasteners, buttons, snaps, zipper) such that the device can be worn. For example, a wearable device can comprise a metal sheet of any size and shape, and can include complementary fasteners on opposite ends or edges of the sheet such that the wearable device can wrap around a portion of a wearer's body and fastened in place. Additionally or alternatively, the wearable device can be incorporated into or fastened to a clothing item (e.g., be placed on an inner surface of a jacket, shirt, pant, or to replace a portion of a jacket, shirt, pant) or an accessory (e.g., hat, helmet) that can be worn by the wearer. For example, it is contemplated that an illumination device of the inventive subject matter may replace or be coupled to overlie a surface of a panel or portion of a clothing or wearable item. In such embodiments, the illumination device can replace or overlie an inner or outer surface of between 1-100%, between 10-75%, between 50-75%, between 10-50%, or any other suitable percentage of the clothing or wearable item.

In some contemplated embodiments, the illumination device can be implantable. In some embodiments, the illumination device can be formed as a container or other housing, or be incorporated into a container, and be configured to provide illumination to an object housed therein, for example, living tissue.

In some contemplated embodiments, the illumination device can be a wearable illumination device sized and dimensioned to be worn over a portion of a body of a wearer that comprises a skin defect and a photosynthetic scaffold placed over or adjacent the skin defect. In some contemplated embodiments, the system can comprise the photosynthetic scaffold.

Contemplated systems can also comprise one or more sensors (e.g., a temperature sensor) placed on or adjacent a wound or living tissue, or attached or otherwise coupled to the metal or other substrate or other components of the system. The sensor can be configured to measure, for example, temperatures of one or more of the electronic driver, the metal or other substrate, the battery circuit and other components, and communicate the data to a control unit configured to control the set of lights. Other contemplated sensors include suitable metabolic, chemical or physiological sensors that could provide additional information to a user (e.g., data relating to a skin temperature, a pH, an electrolyte level, an oxygen level). The one or more sensors can be operatively coupled to a controller such that light emission is controlled or adjusted based on the sensor data (e.g., shut down, adjust an intensity of a light based on, for example, oxygen sensors, adjust a pattern of light, adjust an “off” period). The system can comprise one or more alarms or shutdown mechanisms to ensure that the metal or other substrate and other components do not overheat. For example, if a temperature of the metal or other substrate exceeds a threshold temperature, an alarm can be triggered, or the system (or components thereof) can automatically be powered off to avoid overheating.

Contemplated systems can comprise a control unit configured to be used to adjust an intensity of the set of lighting elements. The control unit can be coupled to the temperature sensor, the alarm, and a display screen that displays relevant information relating to the system (e.g., temperature information, light cycles, intensity, time remaining in a light cycle).

The set of lighting elements can be arranged in any suitable configuration. In some embodiments, the lighting elements can preferably arranged in a rhomboid or hexagonal configuration, which was found to be optimal for the formation of a flat illumination profile while keeping wiring simple.

Other advantages and benefits of the disclosed compositions and methods will be apparent to one of ordinary skill with a review of the following drawings and detailed description.

### BRIEF DESCRIPTION OF THE DRAWINGS

The structure and operation of the present invention will be understood from a review of the following detailed description and the accompanying drawings in which like reference numerals refer to like parts and in which:

FIG. 1 illustrates a laboratory phase and a clinical phase for a study described herein;

FIG. 2 illustrates when blood and biopsy samples were taken for the study of FIG. 1;

FIG. 3 describes the participants of the study of FIGS. 1 and 2, and includes images of participant's wounds;

FIG. 4 illustrates an embodiment of an illumination device of the inventive subject matter;

FIG. 5 illustrates another embodiment of an illumination device;

FIG. 6 illustrates an embodiment of a control unit of the inventive subject matter;

FIGS. 7A-D illustrates LEDs of an illumination device with different intensities;

FIG. 8 illustrates the capacity of an illumination device of the inventive subject matter to induce oxygen production by C. reinhardtii;

FIG. 9 illustrates an illumination device worn on a limb of a wearer;

FIGS. 10A-10D are images from a surgical procedure and the clinical outcome;

FIGS. 11A-11F illustrate results of self-evaluation of patients of the study of FIG. 3;

FIG. 12 summarizes hematological results from the study of FIGS. 1-3, and 11A-F;

FIG. 13 shows serum electrolyte levels evaluated before and during the study of FIG. 3;

FIGS. 14A-14F show Peripheral lymphocytes subpopulations in the serum of patients implanted with photosynthetic scaffolds in the study of FIG. 3;

FIGS. 15A-15F show Cytokine profile in patient plasma samples in the study of FIG. 3;

FIGS. 16A-16D show histological assays performed in the study of FIG. 3; and

FIGS. 17A-17C illustrate histology of photosynthetic control scaffolds.

### DETAILED DESCRIPTION

The detailed description, in connection with the accompanying drawings, is intended as a description of various embodiments and is not intended to represent the only embodiments in which the disclosure can be practiced. The detailed description includes specific details for the purpose of providing a thorough understanding of the embodiments. However, it will be apparent that those skilled in the art will be able to understand the disclosure without these specific details.

The present invention is generally directed towards illumination systems comprising a wearable illumination device, especially for use for treating living tissue. The illumination device can comprise a metal substrate, a living hinge, and a set of lighting elements. The set of lighting elements can be supported by a set of printed circuit boards and connected via a set of cables (e.g., where the lighting elements comprise LEDs). The metal substrate is preferably a copper substrate, but all suitable metals that are malleable and conductors of heat and electricity are contemplated. The living hinge can comprise thinned or cut/cutout portions or any suitable size and shape to allow the more rigid metal sheet to bend along the lines of the hinge. The set of lighting elements can comprise any suitable lighting elements, including for example, light emitting diodes or fiber optic lights.

Insufficient oxygen supply represents a large clinical issue in several fields of human physiology and medicine. It has been suggested that the implantation of photosynthetic cells could provide oxygen to tissues even in the absence of a vascular supply. These photosynthetic therapies have shown to be successful in several in vitro and in vivo models. An early phase-1 clinical trial was recently conducted to evaluate the safety and feasibility of implanting photosynthetic scaffolds for dermal regeneration in six patients with full-thickness skin defects. Exemplary photosynthetic scaffolds, photosynthetic cellular substances and methods of use are described in U.S. patent application Ser. Nos. 15/845,016, filed on Dec. 18, 2017, and 14/845,063, filed on Sep. 3, 2015.

The photosynthetic scaffolds described in the '016 and '063 applications and suitable for use with illumination systems of the inventive subject matter include photosynthetic scaffolds that delivers oxygen and its uses for tissue engineering and the treatment of ischemia. The term “scaffold” is defined broadly to include any structure or carrier matrix to which cells can attach or on which cells can proliferate, and should be interpreted as including sutures embedded in deep layer tissue, a mesh, a bandage, or any other suitable material on which cells can attach or proliferate. The photosynthetic structures, such as photosynthetic cells, algal cells, photosynthetic bacteria, isolated chloroplasts, and cells obtained from vascular plants, used for the scaffold of the present invention can be any type of cells that are able to grow and to be photosynthetically active. In some aspects, the photosynthetic cells may be present in a standalone liquid, gel, or cream. For example, a topical cream can include photosynthetic cells and is applied to the surface of a patient's skin. The photosynthetic cells used according to the present invention are those that are active in the presence of cells derived from different tissues, like dermal, bone and nerve tissue as well as blood tissue. In a preferred embodiment the photosynthetic cells used for the scaffold of the present invention are unicellular algae from the genus Chlamydomonas, in particular Chlamydomonas reinhardtii which can grow and maintain photosynthesis thereby delivering oxygen. Thus, by incorporating the photosynthetic cells or other photosynthetic structures in a scaffold, a “photosynthetic scaffold” is obtained, which can continuously release oxygen, providing the basis for tissue growth and regeneration.

It should be appreciated that the lighting devices described herein, while useful for use with photosynthetic scaffolds, may be used in any suitable devices, systems and methods where light is provided to a person, living tissue or other object.

Study Showing Safe Uses of Illumination Systems and Photosynthetic Scaffolds for Tissue Regeneration in Humans

The trial described herein and conducted on behalf of Applicant shows that the presence of the microalga C. reinhardtii did not trigger any deleterious local or systemic immune responses in a 90 days follow-up, therefore allowing for full tissue regeneration in humans. The results presented strongly support the translation of photosynthetic therapies into clinical use, and contribute to the better understanding of induced therapeutic symbiotic relationships between humans and photosynthetic cells.

In the experiment described below, scaffolds containing high concentrations of microalgae were implanted into human full-thickness skin defects and covered with a light-emitting dressing. Afterwards, tissue regeneration and several other relevant clinical and laboratory aspects were analyzed for up to 90 days. It was demonstrated that photosynthetic microalgae can be safely implanted in human patients, therefore allowing for dermal regeneration. This first in-human trial will contribute to translating photosynthetic therapies into other areas of regenerative medicine, as well as other general fields of biomedical research and physiology.

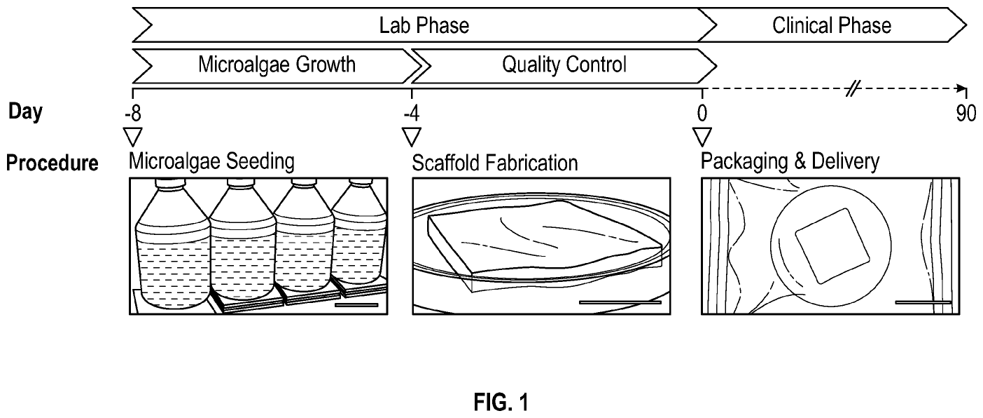

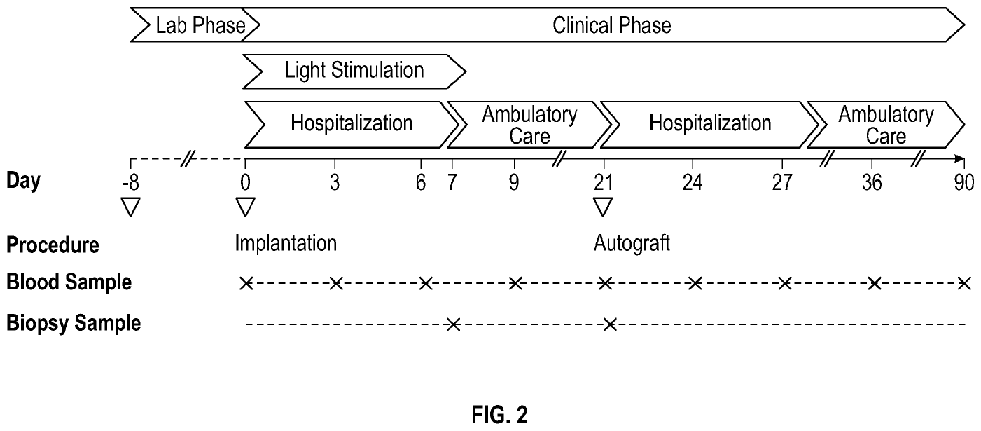

This study was a single-center first-in-human trial to evaluate the safety of implanting photosynthetic scaffolds for dermal regeneration in full-thickness skin defects (ClinicalTrials.gov identifier: NCT03960164). Microalgae (C. reinhardtii) were cultured under sterile conditions, and incorporated in a commercially available scaffold for dermal regeneration as shown in FIG. 1. Four days after seeding, results showed an increase in the scaffold's green color intensity, therefore confirming a high proliferation capacity of the microalgae prior to implantation. The presence of anaerobic bacteria, fungi and rapidly growing mycobacteria were not detected in biopsy samples, and photosynthetic scaffolds were packed and delivered to the hospital to start the clinical phase shown in FIG. 2.

FIG. 1 illustrates the study design. In the laboratory phase, for the photosynthetic scaffold fabrication, C. reinhardtii were cultured for four days under good laboratory practice (GLP) conditions, and a biopsy was taken for quality control. After four additional days, once all microbiology tests were negative, scaffolds were packaged and delivered to the Hospital del Salvador (Santiago, Chile) for immediate use. In the Clinical phase, photosynthetic scaffolds were implanted according to the inclusion and exclusion criteria for this study. After implantation, scaffolds were illuminated for seven days. Then, as shown in FIG. 2, patients were subjected to 14 days of ambulatory care, followed by a conventional autologous partial skin graft over the previously implanted photosynthetic scaffold (day 21). Six days later patients were discharged, and received ambulatory care for up to 90 days. Blood and biopsy samples were taken at the time points indicated in the figure. Scale bars represent 10, 2, and 5 cm (from left to right in FIG. 1).

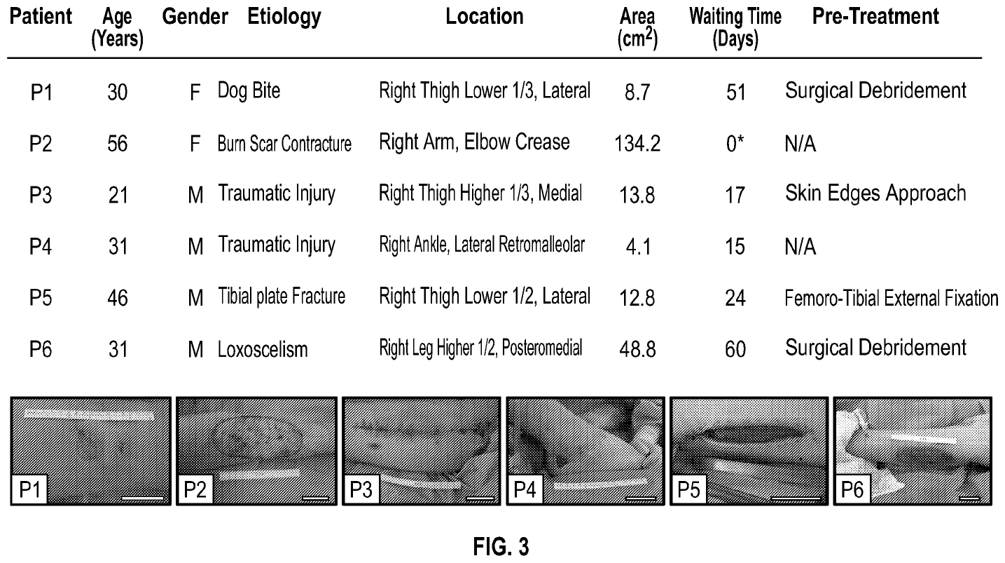

A total of six volunteers (two females and four males) were treated in this study, as shown in FIG. 3. Most wounds corresponded to single full-thickness skin defects, except for one patient (P3 in FIG. 3) who was treated for multiple defects generated in the same accident. The etiology of the wounds, as well as the body location were diverse, but were all located in the extremities. The wound area was highly variable among patients, with a range of 4.1 to 134.2 cm². Additionally, the time between the injury and treatment was variable and fluctuated from 0 to 60 days. It is worth noting that in patient 2 (P2 of FIG. 3), waiting time was considered as 0 days, as the skin defect was created during the same surgical procedure as the scaffold implantation due to scar removal, however, the original lesion (burn scar contracture) was present for several decades in the patient. Finally, the age of treated patients was broad, ranging from 21 to 56 years old, as shown in FIG. 3.

Illumination Device

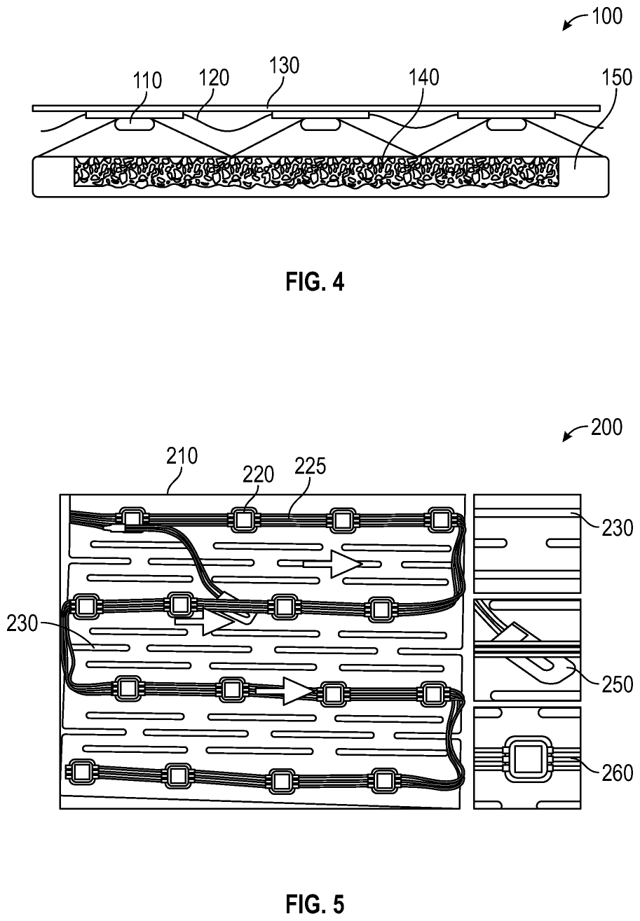

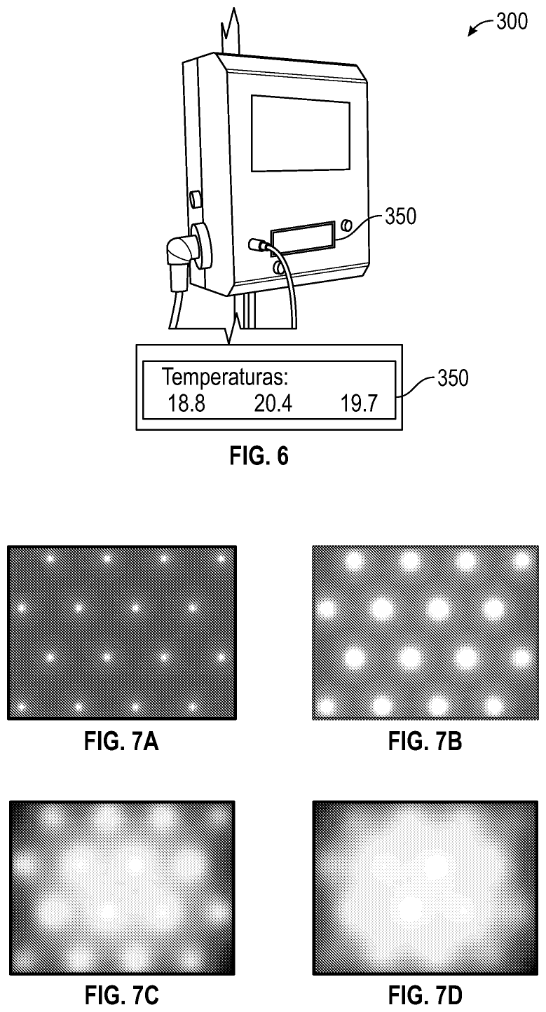

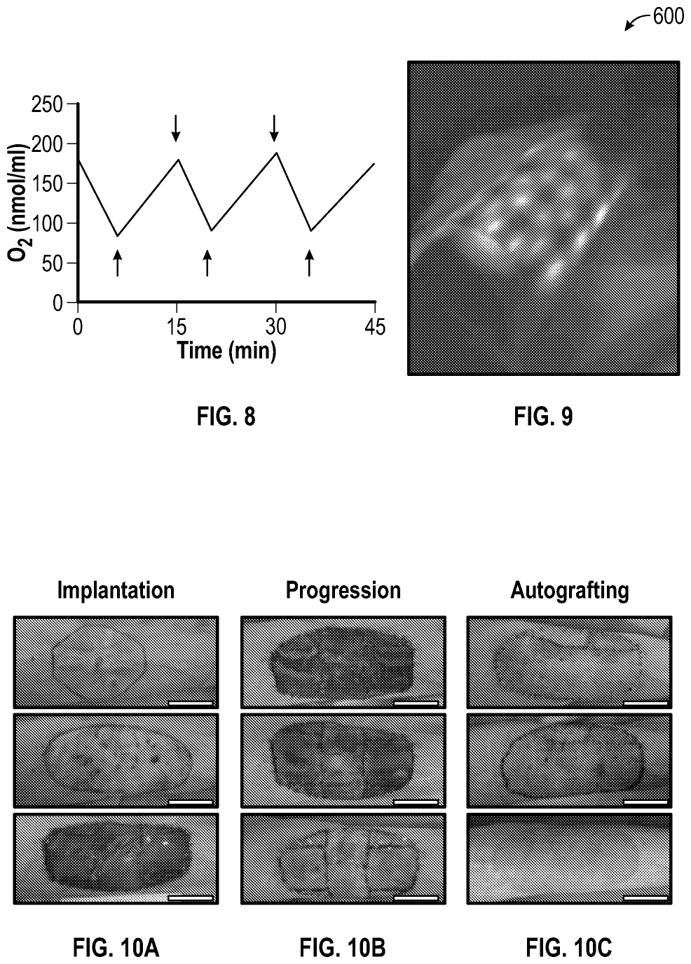

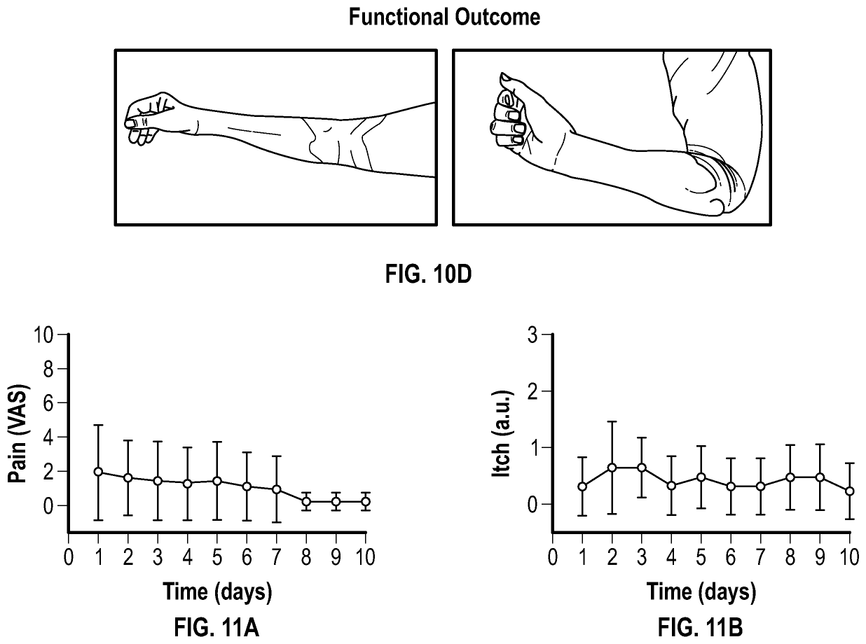

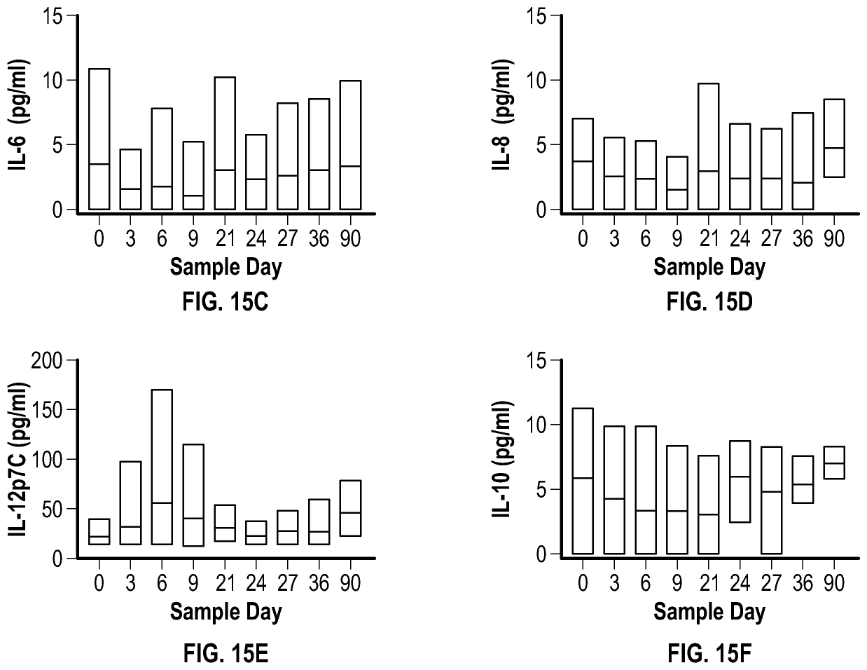

In order to provide light to the implanted photosynthetic scaffold, illumination devices and systems were specially developed as used and shown in FIGS. 4-9. FIG. 4 illustrates a schematic view of the illumination device placed over the photosynthetic scaffold and dermis. FIG. 5 is a representative image of an illumination device. Arrows indicate magnified areas showing: living hinge 230 pattern printed on the copper substrate 210 to allow flexibility where desired, one or more sensors 250, for example, at least one of a temperature sensor, a metabolic sensor and a physiological sensor, and LED lights or other lighting elements. In the example shown, LED lights are supported by a PCB and connected through flat ribbon cables 260. However, any suitable lighting elements are contemplated, including for example, fiber optic lighting elements. FIG. 6 is an image of a control unit 300 placed on a clinical holder for easy and secure handling. This unit includes an LCD screen 350 that serves as a user interface, displaying values for light intensity, battery state, temperatures for the electronics driver, copper substrate, and battery circuit, and sensor data gathered from the one or more sensors. FIGS. 7A-D show images of the LED intensity of a device adjusted through the control unit using pulse-width modulation (PWM), allowing remote control of the LED illuminator. In some contemplated embodiments, the light intensity or other feature of the light can be modified automatically based on sensor data from the one or more sensors. Light intensities correspond to 2% (FIG. 7A), 4% (FIG. 7B), 10% (FIG. 7C) and 15% (FIG. 7D) of the system's maximal power. All suitable light intensities for the devices are contemplated, including between 2-15% intensity, between 2-30% intensity, or any other suitable intensity. For example, it is contemplated that a device of the inventive subject matter can be configured to provide a light intensity that automatically adjusts between 2-50%, between 2-30%, between 2-15%, or any other suitable range, based on sensor data received from one or more sensors. FIG. 8 illustrates the capacity of the illumination device to induce oxygen production by C. reinhardtii was validated in vitro. Lower and upper arrows indicate the time point when the illumination was turned on and off respectively. It should be appreciated that the light can be pulsed and include on/off cycles of, for example, between 1 second and 45 minutes, between 1-minutes, between 5-15 minutes, between 2-30 minutes, or any other suitable cycle times. It should also be appreciated that the on cycles can be for a longer, shorter, or the same time as an off cycle. For example, the light can be pulsed to include on/off cycles where each on cycle is between any of 1 second and 45 minutes, between 1-30 minutes, 1-15 minutes, between 5-15 minutes, between 2-10 minutes, between 1-10 minutes, between 2-8 minutes or between 2-30 minutes, and each off cycle is between any of 1 second and 45 minutes, between 1-30 minutes, 1-15 minutes, between 5-15 minutes, between 2-10 minutes, between 1-10 minutes, between 2-8 minutes or between 2-30 minutes. FIG. 9 shows that the safety of the device was evaluated on a volunteer's arm. Scale bar represents 4 cm in FIG. 5 and 10 cm in FIG. 6.

To reduce heating of the implant, and to ensure homogeneous lighting throughout the whole system, light propagation was modeled to find a geometrical pattern that would produce a flat illumination with a minimal number of LEDs or other lighting elements (e.g., illumination end of optical fiber(s) coupled to LED or other light source(s)). Various sizes were made for the illumination device, in order to fit different wound dimensions. The smallest device was composed of 4 LEDs while the biggest one operated 16 LEDs. However, it should be appreciated that an illumination device can comprise any suitable number of LEDs or other lighting elements (e.g., one powerful LED, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 24, at least 28; between 1-50, between 1-25, between 1-36, between 1-15, between 1-16, or any other suitable number of LEDs or other lighting elements).

On each device, the illumination LEDs followed a rhomboid or hexagonal arrangement as shown in FIG. 5, which was found to be optimal for the formation of a flat illumination profile while keeping wiring simple. Each lighting element (e.g., LED, set of LEDs, fiber optic illumination ends, set of fiber optic illumination ends where the light is emitted) can be spaced apart from an adjacent lighting element as appropriate to induce local photosynthesis. To facilitate dissipation of the local heat produced by the high-powered LEDs, they were attached to a highly thermal-conductive copper sheet. In the illustration of FIG. 4, device 100 comprises a copper sheet 130 having a set of LEDs 110 disposed on a first side, wherein the LEDs are connected by cable 120. The device 100 was placed over an injured dermis 150 implanted with a photosynthetic scaffold 140. It should be appreciated that the device 100 can be placed directly over the injured dermis and photosynthetic scaffold, or can be placed over it to provide a gap between the LEDs of the device 100 and the dermis and/or scaffold. The gap can be of any suitable distance(s), including for example, a gap of less than 2 cm, less than 1 cm, at least 1 cm, at least 2 cm. The device can be configured with spacers (e.g., padding adhered to the surface of the copper sheet on the same side as the LEDs) to provide a gap between the LEDs/device and the dermis/scaffold. Each spacer can have any suitable length (extending across a surface of the metal or other substrate) thickness (extending between the metal or other substrate and the dermis and/or scaffold), including for example, a length of between 1-10 cm, between 1-5 cm, between 1-3 cm, between 0.5 and 2 cm, between 0.5 and 1 cm, and thickness of 1-10 cm, between 1-5 cm, between 1-3 cm, between 0.5 and 2 cm, between 0.5 and 1 cm.

In some contemplated embodiments, the device can be wearable and incorporated into a wearable item such as a vest, a legging, an arm band, a leg band, an ankle band, a knee band, a neck band, a helmet, a face mask, or any other suitable item. In some embodiments, the illumination device can be formed as a container or housing, or be incorporated into or coupled to a housing. The illumination device can be configured to provide illumination to an object housed therein, for example, living tissue. In some embodiments, the illumination device can comprise or be positioned in a container such as a cooler for storing an organ or other tissue that is being transported to a location for implanting into a patient for a medical procedure. The container can be fitted with the illumination device to illuminate the interior space of the container, and the container can be filled with a scaffold, liquid, gel or other substance that includes photosynthetic cells that produce oxygen in the presence of light, and help maintain the tissue or organ until it can be transplanted. In some contemplated aspects, an organ or other tissue can also be perfused with photosynthetic cells to increase the supply of oxygen to the interior portion of the organ or tissue, and an illumination device of the inventive subject matter can house, surround, or be placed directly on or in close proximity to the organ. In some contemplated embodiments, the illumination device can be used inside a patient's body as an internal device to illuminate an implant comprising photosynthetic cells. In some contemplated embodiments, the illumination device can be used for inner illumination (e.g., as an organ preservation device, or for wounds that are located in other tissues.

Constant measurements of the temperature or other metrics were provided by a sensor attached or otherwise coupled to the control unit. To add more flexibility to the copper plate, a living hinge pattern was machined, which allowed the device to adapt to the patient's different anatomical requirements as shown in FIG. 5. As illustrated in FIG. 5, a copper plate 210 includes a set of LEDs 220 connected by cable(s) 225. Living hinges 230 are created via thinned portions and/or cutouts. In the embodiment shown, the living hinge is created by sets of cutouts, wherein a middle row of cutouts includes cutouts that are offset from cutouts of adjacent rows (the row above and below it). The cutouts of the upper and lower rows can be vertically aligned, while the cutouts of the middle row are not vertically aligned with any of the cutouts of the upper or lower rows (although there may be overlap). Is it contemplated that a metal or other substrate of the devices and systems described herein can comprise any suitable living hinge pattern(s) including, among others, a bastian pattern, a bowling pin pattern, a brackets pattern, a cross pattern, a diamond patter, a straight pattern, a triangle pattern, a beehive pattern, a wave pattern, and/or any other known or later discovered living hinge patterns.

The control unit 300 of FIG. 6, which can be coupled to the device and the one or more sensors, can be configured to supply energy to the illumination device, and to display relevant information such as temperatures of the electronic driver or a user's skin, copper plate and battery circuit, as well as light cycles and intensity settings (as shown in FIGS. 7A-D). Additionally, the unit can be equipped with alarms and a shutdown security system to ensure that the illumination plate does not overheat. Tests were performed to confirm that the LED plate did not heat up significantly when the light intensity was kept under 50%, which was previously confirmed to be above the required radiation. Once the device was proven safe in laboratory conditions, its capacity to induce photosynthesis in vitro was evaluated. Here, oxygen production was immediately detected upon light stimulation of the microalgae C. reinhardtii, while oxygen consumption was observed in the absence of light (FIG. 8). Finally, safety and wearability of the device were tested in two healthy subjects exposed for 8 hours to 50% light intensity proving the feasibility of its use in patients. FIG. 9 illustrates a device of the inventive subject matter worn on a limb of a patient. The device can be worn on a portion of a limb comprising a skin defect and a photosynthetic scaffold placed over of adjacent the skin defect. It is contemplated that devices can be sized and dimensioned to be worn on certain body parts (e.g., an arm, a leg, a chest), and that the device can include a fastener (e.g., hook and loop, snap(s), zipper(s), adjustable straps with hook and loop fasteners) to secure the device in place.

Although the devices described herein are directed primarily to devices including LED lighting components (including Micro-LEDs, organic LEDs), it should be appreciated that other suitable lighting components can be used (e.g., optical fibers with any suitable light source(s)). Optical fiber or bundles of optical fibers having illumination or light emitting portions that act as a set of lighting elements for the devices contemplated herein can be beneficial in avoiding or reducing the local heat produced by LED-based devices.

Photosynthetic Scaffold Implantation and General Clinical Outcome

FIGS. 10A-10D show images from a surgical procedure and the clinical outcome. FIG. 10A shows a burn contracture scar in the cubital fossa was resected, generating a full-thickness skin defect which was covered with the photosynthetic scaffold. FIG. 10B shows that, after surgery, a change in the color of the wound was observed on days 1, 7 and 21 post implantation (upper, middle and low, respectively). FIG. 10C shows, on day 21 post surgery, the silicon layer of the scaffold was removed and the split skin autograft was fixed to cover the wound. Excellent integration between neodermis and the autograft was observed, as shown for days 27 (middle) and 90 (lower). At all times, no local signs of inflammation were observed in the contact zone between the photosynthetic scaffold and healthy skin. FIG. 10D shows, that 90 days after implanting the photosynthetic scaffold, a functional recovery of the wound was observed as the patient could fully extend and flex the elbow. Scale bars represent 5 cm.

All wounds of patients in the study were full-thickness defects and were comparable; however, their heterogeneity had an impact on the detailed surgical procedure, treatment, and clinical outcome. As an example, the complete procedure described in FIG. 1 is shown for a single patient (FIG. 10; P2 in FIG. 3). Here, a burn scar contracture was removed from the cubital fossa, generating a fresh, clean and larger skin defect, which allowed full elbow extension (FIG. 10A upper and middle). Five scaffolds (5×5 cm² each) were placed directly over the wound, and surgical sutures were used to fix the scaffolds among them and to the wound edges (FIG. 10A lower). A similar procedure was used for P3 and P6 in FIG. 3, where several scaffolds were used, however a single scaffold was enough to cover the entire defect in P1, P4 and P5. Immediately after implantation, blood infiltration was observed in the scaffolds, increasing over the following days (FIG. 10B upper and middle). Twenty-one days after implantation, adequate adherence to the wound bed and vascularization of the scaffold was observed, as well as the presence of only a few spots of free microalgae and erythrocytes (FIG. 10B lower). Notably, the contact zone between the photosynthetic scaffold and the healthy skin did not show clinical signs of inflammation at any time and in any patient, even though scaffolds were loaded with high concentrations of photosynthetic microalgae. As was planned in advance, four out of the six patients (P1, P2, P5 and P6 in FIG. 3) went to autologous skin grafting, and results show its full integration with the neodermis, and no signs of morbidity (FIG. 10C). After the whole procedure, the regenerated skin showed an excellent functional outcome, evidenced by the full mobility recovered in the patient's arm (FIG. 10D).

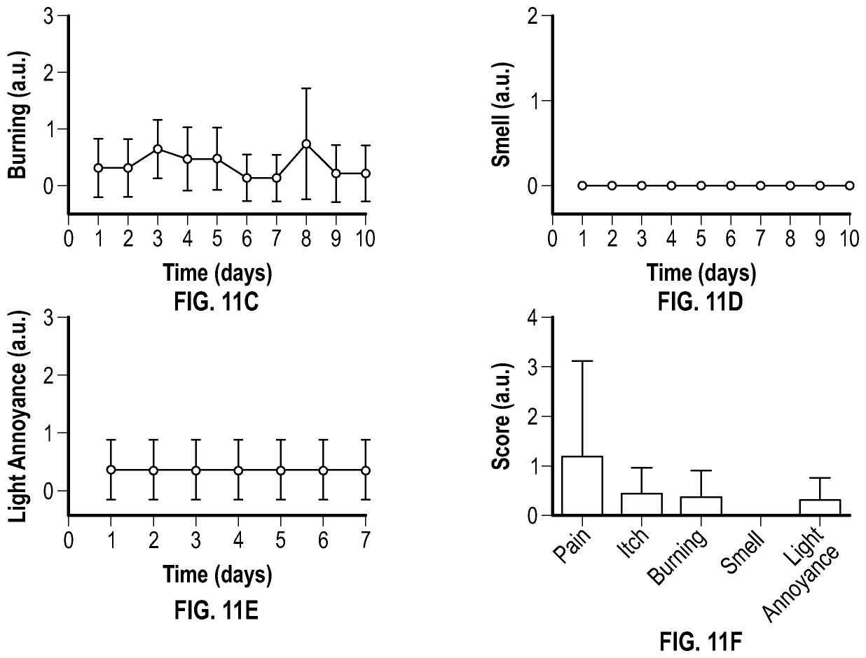

In addition to the clinical outcome described above, self-evaluation of the patients did also support the safety and feasibility of this photosynthetic approach as shown in FIGS. 11A-F. Patients conducted daily self-evaluation for multiple parameters. After implantation of the photosynthetic scaffold several key relevant parameters were evaluated by the patients for 10 days. FIG. 11A represents the pain intensity is expressed by the 0-10 visual analog scale (VAS). Proportional Likert scales (arbitrary units) were used for FIG. 11B itching (0-3), FIG. 11C burning (0-3), FIG. 11D smell (0-2) and FIG. 11E light annoyance (0-3). FIG. 11F represents values for each parameter and all patients during the entire evaluation period. Kruskal-Wallis test evaluated of different parameters, and no significant differences were observed. Data represented as the mean value±SD.

Five clinically relevant parameters were evaluated for up to 10 days after photosynthetic scaffold implantation. Visual analog scale (VAS) for subjective pain intensity (proportional 0 to 10 range) showed no pain (VAS≤1) reported in four patients (P1, P3, P5, and P6 in FIG. 3) during the whole evaluation period, while one patient (P2 in FIG. 3) reported mid to high pain score (VAS=6) at the first postsurgical day, which progressively decreased towards day 7. Another patient (P4 in FIG. 3) reported persistently mid pain scores (VAS=6) during the 7 postsurgical days, and was unable to identify if the pain source was the subjacent calcaneus bone fracture or the implanted skin wound. Moreover, after a four months follow up, this patient still reports pain from the healed bone. Almost all patients reported no or low itch sensation in the implanted wounds (FIG. 11B). Interestingly, the mid score was declared by one patient on the second postsurgical day (P2 in FIG. 3). Burning sensation at the implantation site was reported as absent or low (scores 0 or 1) during the whole evaluation time, nevertheless, patient P2 reported a transient mild burning sensation at day 8 post surgery. (FIG. 11C). None of the patients reported any particular smell from the photosynthetic scaffold during the evaluation period (FIG. 11D). Only ⅓ of the patients (P3 and P5) reported a low light annoyance generated by the illumination dressing during all evaluation days, while the rest of the patients reported it as nonexistent (FIG. 11E). A summary of the self-evaluated scores for each parameter is shown in FIG. 11F.

Systemic Response After Scaffold Implantation

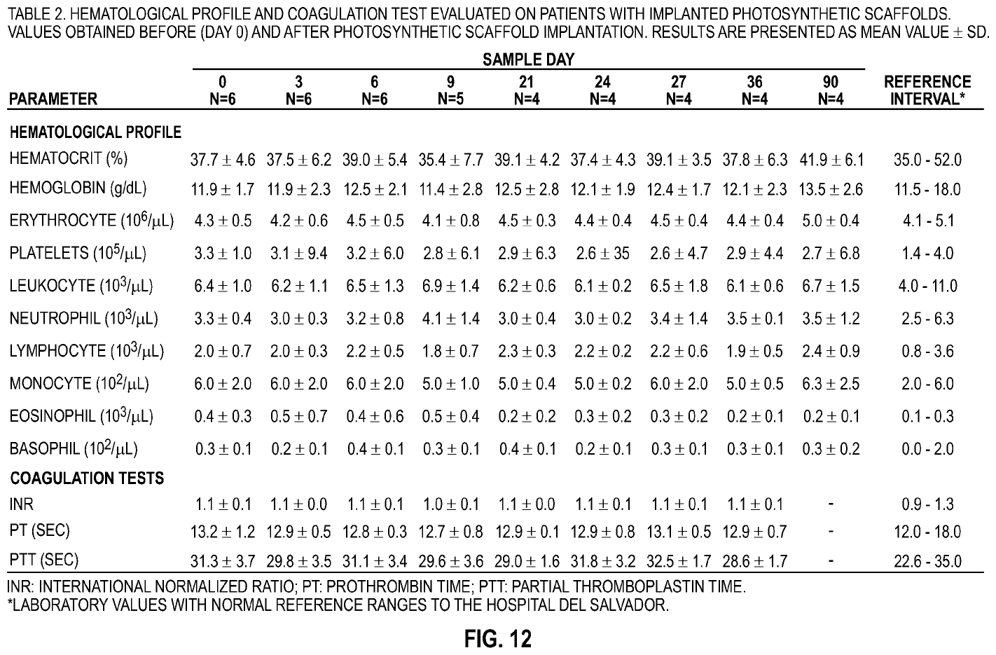

Towards evaluating systemic responses to the wound treatment, blood samples were taken at different times, and critical parameters were assessed for up to 90 days, as shown in FIG. 12. However, some tests were not reported for some patients due to institutional clinical laboratory coordination or patient partial desertion. Hence, the number of analyzed samples decreased as: n=6 from days 0 (pre-implantation) to 6, n=5 at day 9, and n=4 from days 21 to 90. From the hematological point of view, no significant differences were observed in the mean values of hematocrit percentage, hemoglobin concentration and erythrocytes count (FIG. 12). Similarly, the number of platelets did not vary along with the treatment. Non-significant changes were detected in the total number of leukocytes nor in specific subpopulations of neutrophils, lymphocytes and basophils. All the parameters mentioned above were found normal within the institutional reference values during the 90 days follow-up. However, eosinophil counts were over the limit from days 0 to 9, but became normal in the following sampling times (FIG. 12). Besides, coagulation tests were performed (FIG. 12), including international normalized ratio (INR), prothrombin time (PT) and partial thromboplastin time (PTT), which did not show significant changes compared to photosynthetic scaffold preimplantation (day 0). Hematological results are summarized in FIG. 12.

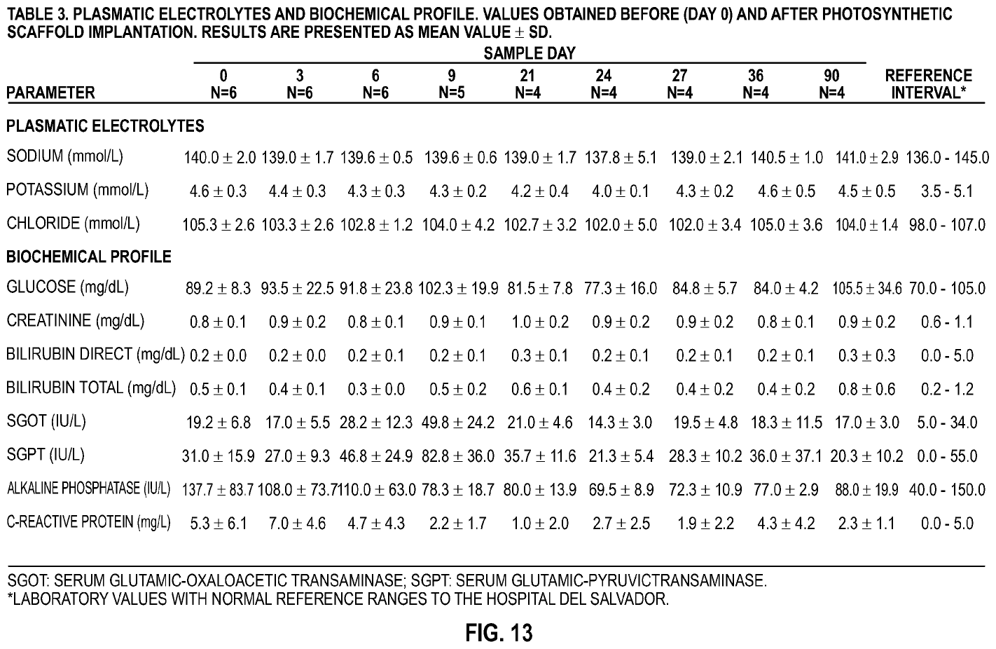

Serum electrolytes, i.e., sodium, potassium and chloride were evaluated before (day 0) and up to 90 days, post-implantation of the photosynthetic scaffold, as shown in FIG. 13, and no significant changes or values outside the institutional reference range were detected during all the evaluation period. The biochemical profile tested metabolic (blood glucose), renal function (creatinine), hepatic function (bilirubin direct and total, serum glutamic-oxaloacetic transaminase (SGTO), serum glutamic-pyruvic transaminase (SGPT), and alkaline phosphatase, bone metabolism (alkaline phosphatase), and inflammatory acute phase response (C-reactive protein) parameters during the evaluation period (FIG. 13). Once again, no changes nor values outside institutional reference ranges were detected, except for C-reactive protein which shows high values from day 0 (pre-scaffold implantation) and days 3 and 6 post implantation, which decreased on days 9 and 21 within normal institutional reference ranges (FIG. 13).

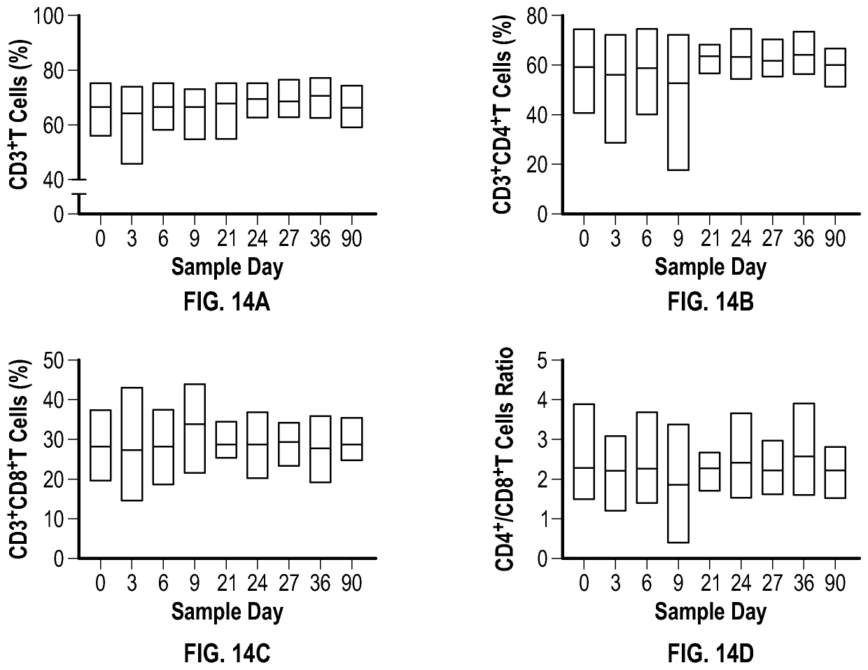

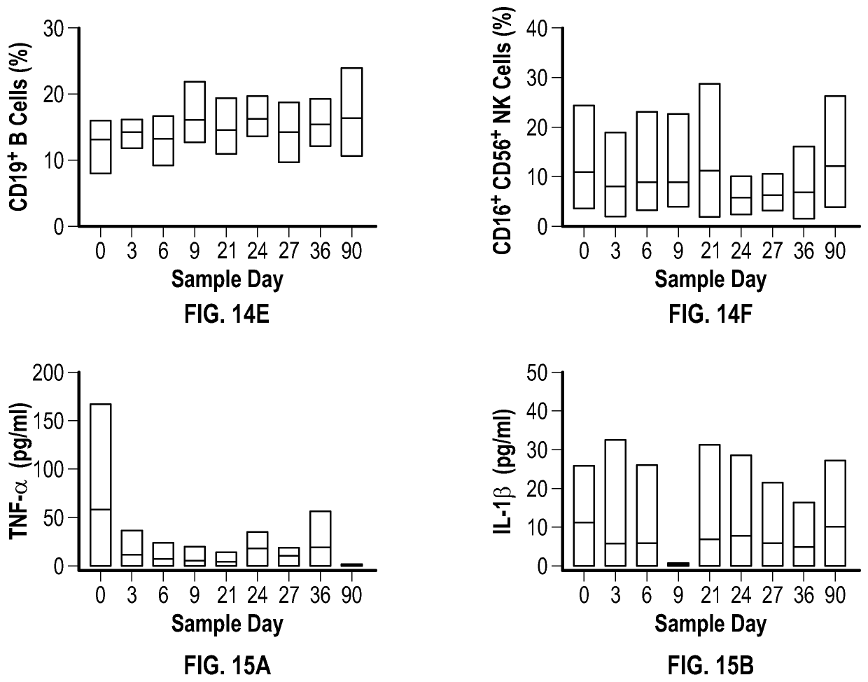

The systemic inflammatory response to the photosynthetic scaffold was analyzed in detail. Hence, lymphocyte composition, as well as the concentration of inflammatory cytokines were evaluated. Total percentage of T-cells (CD3+), T-helper cells (CD3+CD4+) and cytotoxic T-cells (CD3+CD8+) or their ratio (CD4+/CD8+) did not increase after surgical procedure, nor during further times as shown in FIG. 14, showing that the photosynthetic implant did not trigger a Th1 or Th2 immune responses.

FIGS. 14A-14F show Peripheral lymphocytes subpopulations in the serum of patients implanted with photosynthetic scaffolds. Lymphocytes were evaluated before (day 0) and after scaffold implantation. (FIG. 14A) Total of T-cells (CD3+), (FIG. 14B) T-helper cells (CD3+CD4+), (FIG. 14C) Cytotoxic T-cells (CD3+CD8+), (FIG. 14D) ratio (CD4+/CD8+), (FIG. 14E) B-cells (CD19+) and (FIG. 14F) NK cells (CD16+CD56+). Results are expressed as the percentage of cells for each phenotype. The top of the box represents the maximum value, the bottom of the box represents minimum value, and the solid line in the middle represents the mean. Kruskal-Wallis test evaluated lymphocyte populations and no significant differences were observed. Day 0, 3, 6 and 9 (N=5); day 21 (N=4); day 24, 27, 36 and day 90 (N=3).

The same results were obtained for B-cells (CD19+) and NK cells (CD16+CD56+), confirming that the Th2 immune response was absent. Comparable results were observed for six circulating inflammatory cytokines (TNF-α, IL-β, IL-6, IL-8, IL-10, and IL-12p70) where no significant changes could be detected as a consequence of the photosynthetic treatment, as shown in FIGS. 15A-15F.

FIGS. 15A-15F show Cytokine profile in patient plasma samples. The concentration of inflammatory cytokines was determined before (day 0) and after photosynthetic scaffold implantation. (FIG. 15A) Tumor necrosis factor alpha (TNF-α), (FIG. 15B) Interleukin-1 beta (IL-β), (FIG. 15C) Interleukin-6 (IL-6), (FIG. 15D) Interleukin-8 (IL-8), (FIG. 15E) Interleukin-12 (IL-12p70) and (FIG. 15F) Interleukin-10 (IL-10). The top of the box represents the maximum value, the bottom of the box represents minimum value, and the solid line in the middle represents the mean. Kruskal-Wallis test evaluated cytokine secretions, and no significant differences were observed. Day 0, 3, 6 and 9 (N=6); day 21 (N=5); day 24, 27 and 36 (N=4) and day 90 (N=3).

Local Response After Scaffold Implantation

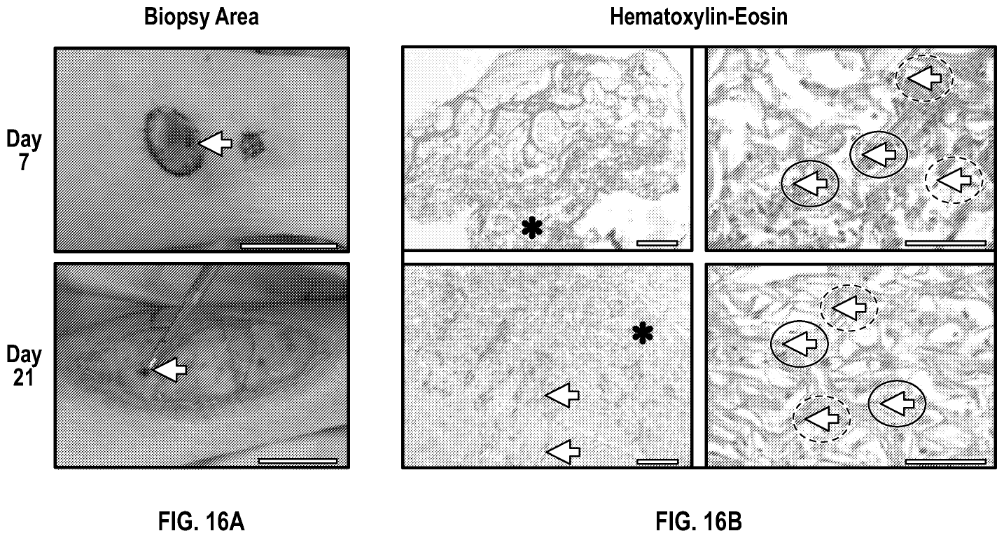

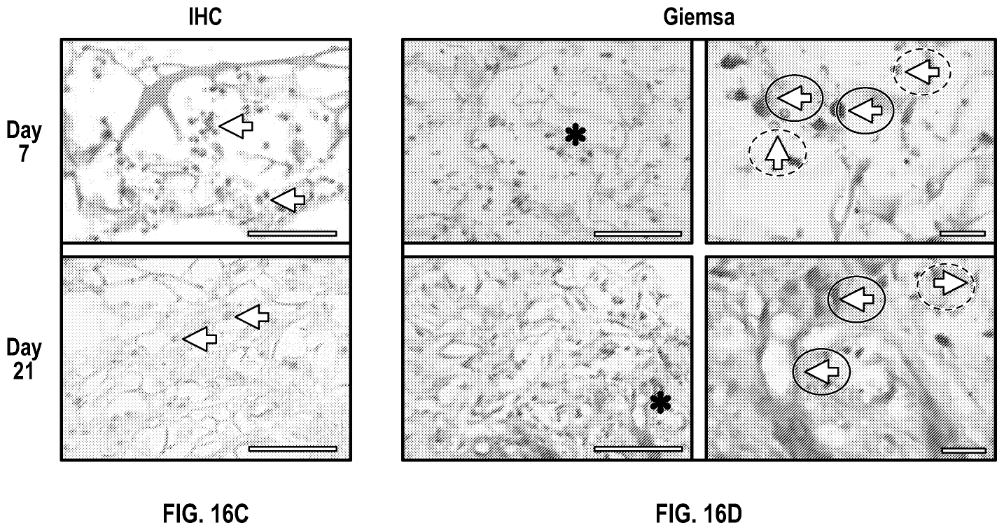

In order to determine the local effect of the photosynthetic scaffold, histological assays were performed as shown in FIGS. 16A-16D.

Histology of implanted photosynthetic scaffold. As shown in FIG. 16A, biopsy samples (arrows) were taken at day 7 and 21 post implantation. As shown in FIGS. 16B, Hematoxylin-Eosin stain shows the presence of collagen fibers, fibrin deposition and infiltration of fibroblast (arrows circled in right two images with dotted lines) and immune cells (arrows circled in right two images with solid lines) at day 7. In day 21, shows multiple blood vessels (arrows in left two images) oriented towards the photosynthetic scaffold. As shown in FIG. 16C, Immunohistochemistry shows that macrophages (arrow, CD68 antibody) were also present in the scaffold close to dermis in both days. As shown in FIG. 16D, Giemsa stain shows fibrin, randomly oriented collagen fibrils, erythrocytes (dotted arrows in top right) and microalgae (solid arrows in top right) at day 7. In day 21, fibroblasts (dotted arrows in bottom right) and newly formed blood vessels (solid arrows in bottom right) were found in the scaffold. Scale bars represent 5 cm (FIG. 16A), 200 μm (FIG. 16B, left), 100 μm (FIG. 16B, right; 16C, 16D left) and 20 μm (FIG. 16D, right). Asterisks in left pictures indicate the magnified area shown in right.

For P1, on day 7 after scaffold implantation, hematoxylin-eosin (H-E) staining of the biopsy allows clear identification from top to bottom of the implanted photosynthetic scaffold, at the dermis (FIG. 16B upper). Higher magnification of the biopsy (FIG. 16B upper right) reveals randomly oriented collagen fibrils with some fibrin deposits, immune cells and fibroblastic cells with scarce cytoplasm and heterochromatic, flattened or spindle-shaped nucleus. Macrophage cells (CD68) presenting a larger morphology did not show intracellular brown residues suggestive of lysosomes (FIG. 16C upper). Fused macrophages, foreign body giant cells or monocytes were not detected. Neutrophils and lymphocytes were occasionally observed and scattered found at the dermis and the scaffold. Infiltrates of any kind (focal, diffuse, superficial or perivascular) were not observed. Giemsa staining confirmed the presence of microalgae (FIG. 16D upper right) showing typical size, morphology and bluish granular cytoplasm. Additionally, erythrocyte infiltration was also observed throughout the biopsy sample.

Hematoxylin-eosin staining of the biopsy sample taken on day 21 before scaffold implantation in P6 (FIG. 16B lower) reveals the presence of cells and vascular structures oriented towards the photosynthetic scaffold. Higher magnification of the biopsy (FIG. 16B lower right) reveals randomly oriented collagen fibrils with some fibrin deposits, fibroblastic cells and immune cells. Specific immunostaining (CD68) performed on this biopsy (FIG. 16C lower) showed the presence of macrophages, and no images for fused macrophages, foreign body giant cells or monocytes were detected. In Giemsa staining (FIG. 16D lower), it is possible to observe some of the characteristics described on day 7, especially those related to the random distribution of the scaffold's collagen fibrils. Additionally, there is an increased amount of extracellular matrix, and distribution of blood vessels shaping a normal dermic histoarchitecture. Higher magnification of Giemsa staining (FIG. 16D lower right) did not show the presence of microalgae, however neovascularization oriented towards the photosynthetic scaffold can be observed, with the presence of multiple early endothelial cells embedded in normal extracellular matrix and moderate presence of spindle-shaped fibroblasts.

It is broadly described how oxygen plays a key role in most steps involved in wound healing. Among others, it is required for energy production, as a signal transduction molecule and antibiotic. Thus, poor oxygenation severely impairs wound healing, being reported as one of the main causes of non-healing in chronic wounds. Lighting has been broadly used in several medical applications, such as photodynamic therapy, psoriasis and hyperbilirubinemia phototherapy where illumination is performed only for short times, specific wavelength, and using well-described protocols. Hence, aiming to induce local photosynthesis, and to discard potentially phototoxic effects, the feasibility of illuminating wounds for extended periods under safe and controlled conditions was tested. In order to stimulate the photosystem II, a novel LED-based device (as shown in FIGS. 4-9) was designed with an emission wavelength of 455 nm, or between 405-505 nm or 450-475. Red light (623 nm) is also absorbed by chlorophyll, and it is contemplated that light of any suitable wavelength (e.g., between 380-740 nm, between 390-700 nm, between 435-475 nm, between 435-650 nm, between 450-475 nm, between 650-675 nm, between 400-700 nm, a combination thereof (e.g., some lights between 450-475 nm, others between 650-675 nm)) can be emitted using a device of the inventive subject matter. Red light may not be preferred as it may increase local heat, which could have a deleterious impact in both, the viability of the microalgae in the wound, and the regeneration process itself. Nevertheless, at high light intensities, noxious temperature was avoided by the high thermal-conductive property of the copper sheet in the LED-dressing, which is continuously monitored by the control unit. New generation of illumination devices could include different integrated chemical and metabolic sensors to control light emission. For instance, desired oxygenation levels could be spatiotemporally achieved by coupling lighting intensity to local oxygen sensors in the wounds.

After implanting photosynthetic scaffolds, relevant negative effects were not reported during the patient's self-evaluation. However, for the few issues reported, it is difficult to dissect the effect of the photosynthetic therapy itself from the whole wound context; nevertheless, the presence of the microalgae in the scaffold did not exacerbate any of the studied parameters. Although to evaluate the efficacy of photosynthetic therapy was out of the scope of the study described herein, a similar analysis is also valid for the regenerative process, where the overall contribution of the photosynthetic oxygen itself was not analyzed. However, it was clear that the presence of the microalgae allowed key regenerative processes such as cell migration, ECM deposition, and vascularization. Potential adverse reactions induced by the photosynthetic scaffold were evaluated both locally, by multiple clinical and histological techniques, and systemically, by analyzing peripheral blood cells and inflammatory blood markers. Increased peripheral thrombogenicity expressed by hematocrit changes, platelet and red blood aggregation at the implant site or changes in the coagulation parameters were completely absent in all patients. The effects of possible systemic infused microalgae by-products on hepatic and renal functions but also on general patients' metabolism were not detected even after 90 days of follow-up.

It is noteworthy to remark that all patients included in the present study were surgically treated for full-thickness skin defects under general anesthesia. Such interventions are associated with increased concentrations of C-reactive protein, which usually reach a peak two or three days after surgery, and are proportional to the intensity and extension of the wound and surgical treatment thus, explaining the increased values of this marker, especially at day 3 post scaffold implantations.

Additionally, the percentage of immune cells in patient's blood serum were evaluated, in response to the photosynthetic scaffold's implantation. T-cells are the cornerstone of the adaptive immune system and play an essential role in the host defense against microbial pathogen. In patients with severe Gram-negative bacteria sepsis, it has been observed early CD4+-lymphopenia and increased CD56+ NK cells compared to control patients. Quantitative differences in the total and subpopulations counts of white blood cells is a well-known parameter to detect immune cellular responses during exposition to biomaterials. Typically, adverse reactions are characterized by an increased count of CD3+, CD4+ and T cells, such changes were not detected in none of our treated patients. Hypersensitivity reactions concerning the collagen component of the photosynthetic scaffold especially that not of human origin. All subtypes of collagen, especially non-human ones, are potent antigens, able to trigger T-cells mediated type IV reactions. Nevertheless, no such reaction or T-cell subpopulation changes were seen in the patients treated. In contrast, the results showed that the implantation of photosynthetic scaffolds did not trigger significant changes in circulating lymphocyte subpopulations compared to values before scaffold implantation.

Cytokines are a group of proteins involved in various biological processes, including growth, differentiation, cell survival, inflammation, apoptosis, necrosis, and fibrosis. Evaluation different cytokines in patients in the acute phase of sepsis showed statistically significant difference versus healthy controls. Critically ill patients showed high levels of IL-1β (about 70 pg/ml), IL-6 (about 36 ng/ml), IL-8 (about 6 ng/ml), and IL-10 (about 2.2 ng/ml) on day 1. Despite the high inter-individual variability in cytokine production by several different pathogen-related stimuli and increase in 3 to 4 orders in magnitude from the baseline values, none of the treated patients showed significantly different concentrations compared to pre-implantation. On the other hand, systemic concentrations of cytokines in humans after plastic surgery trauma (reduction mammoplasty) showed significant increase of IL-6 plasma levels (about 80 pg/ml), on the day of surgery compared with pre-operatory levels (average values are 3.13-12.5 pg/ml), and remained elevated for 7 days. While IL-8 plasma levels remained within the normal range (0-40 pg/ml). The results did not show significant changes in plasma cytokines levels compared to values before surgery and photosynthetic scaffold implantation. This fact strongly suggests that the photosynthetic scaffold did not trigger humoral immune responses.

One of the most intriguing results of this trial was the low local and systemic immune responses observed against the foreign microalgae. Macroscopically this was clear at the wound edges where no clinical signs of inflammation, such as edema or erythema, were detected at any time point. Furthermore, histological results showed an excellent integration between the photosynthetic scaffold and the wound bed. Scaffolds are supposed to serve as temporary matrices for the infiltration of fibroblasts, macrophages and capillaries from the wound bed. New endogenous tissue formation would simultaneously occur with scaffold degradation, ensuring proper regeneration of the tissue. At day 7, the collagen structure of the implanted scaffold could be clearly observed, with the presence of coexisting microalgae and infiltrated cells presenting large cytoplasm and euchromatic nucleus, suggestive of active fibroblasts. Additionally, erythrocytes and infiltrated macrophages were also detected in the implanted scaffold, which are key regulators in the wound healing process. At day 21, microalgae were not observed histologically in the biopsies, which was expected based on our previous reports. However, several tissue regeneration characteristics were consistent with that seen in granulation tissue formation: blood vessels derived from the wound bed were directed towards the implanted scaffold, and an increase in fibroblast infiltration and endogenous collagen deposition were observed. Moreover, macrophages were still present 21 days after scaffold implantation, probably removing the by-products of regeneration, as no fused macrophages nor foreign body giant cells or monocytes were detected in any patient biopsy. These characteristics correspond to those expected in skin wound reparative processes, and are critical for further successful autographing.

Consequently, no rejection of the autograft placed above the photosynthetic scaffold was observed in the four patients that require such surgical procedure. This is interesting when compared to allografts, which have a limited persistence in vivo, as immune rejection usually occurs within a few days or weeks in the case of patients with suppressed immune response or even less in the case of pig xenografts, leading to destruction of the graft carried out predominantly by CD8+ T cells. The immune tolerance observed here to C. reinhardtii, agreed with our previous results reported in zebrafish and mice, and was consistently evidenced by different means in all subjects of this study at all the analyzed time points. Fibrin coating of the algae can only partially explain this result. Due to its half life being estimated in about 10 to 14 days in vivo, a later reaction could have been expected in any case. In contrast to other microorganisms, C. reinhardtii have no pathogenic or toxicogenic potential, and have been granted with a GRAS (Generally Recognized As Safe) status by the US FDA. In fact, the critical pathogen associated molecular patterns that are recognized by the native immune system (e.g. LPS or single strain RNA) have not yet been described to be present in C. reinhardtii. Therefore, an interesting option to consider is that our immune system could not have evolved in the need to recognize such kind of cells as foreign entities. This particular feature of immune tolerance should be explored in more detail, and could have an impact in other medical fields, where these photosynthetic cells could be delivered into the tissues to locally provide oxygen, and also other therapeutic molecules such as recombinant proteins.

Overall, the results presented here show that the presence of microalgae is safe for humans and did not impair the scaffold-based dermal regeneration process. This result could be extremely relevant for the translation of photosynthetic therapies from bench to bedside, but further studies need to be done in order to confirm this in a larger population of patients. Moreover, before testing efficacy, the safety of photosynthetic biomaterials should be tested in hypoxic chronic wounds, which are normally present in compromised patients. Additionally, the safety of genetically engineered microalgae should also be tested to further evaluate potential synergistic effects between the simultaneous release of oxygen and recombinant therapeutic agents that may contribute to wound healing. Since clinical outcomes may vary between individuals, pathologies, or tissues, and studies for efficacy will have to consider the optimization of crucial aspects such as algae density, type of injury and illumination settings.

Altogether, the results presented will significantly aid in the transition and translation of photosynthetic therapies into clinical settings, and contribute to the understanding of potential symbiotic relationships between humans and photosynthetic cells. This novel concept is intriguing and could have enormous translational applications, with an impact far beyond tissue engineering and regeneration. For instance, to adapt human physiology to live in poor oxygen environments like outer space.

Study Design

This study is a single-center, first-in-human, early phase 1 clinical trial with microalgae (ClinicalTrials.gov identifier: NCT03960164) to assess the safety of photosynthetic therapies for the treatment of full skin defects. The clinical trial protocol was previously approved by the Research Ethics Committee of the Hospital del Salvador and the Metropolitan Health Service (RE: 2976 Jul. 7, 2015; SSM, Santiago, Chile).

Patients between 18 and 65 years old with full thickness skin defects and without comorbidities were recruited by the Department of Plastic Surgery at Hospital del Salvador (Santiago, Chile). Exclusion criteria included the following comorbidities: hypertension, diabetes mellitus, chronic liver damage, autoimmune diseases, neoplasia, immunosuppression, coronary heart disease, occlusive arterial disease, chronic smoking, drug and alcohol abuse. Patients with psychiatric disorders, or those suffering from any acute pathology other than the tissue damage, or patients with injuries in the face and/or neck, were excluded. All patients involved in the study signed the informed consent form before inclusion in the study. FIG. 3 summarizes the characteristics of each patient included in the study.

Photosynthetic Scaffold Fabrication and Quality Control

Cell-wall deficient cw15-30-derived UVM4 C. reinhardtii strain was grown photomixotrophically at 20° C. in sterile liquid Tris-Acetate-Phosphate medium (TAP) with constant agitation, and always kept in an exponential growth phase. For light stimulation, a lamp with the full spectrum of white light was used to provide continuous light exposure (2500 1×, eq. 72.5 μE/m²·s). For the photosynthetic scaffold fabrication, Integra matrix bilayer skin (Integra®, Integra Life Science Corporation) was used. Before cell seeding of C. reinhardtii, 25 cm² scaffolds were slightly dried on a sterile gauze, and placed silicone-face down on a sterile cell culture plate. Then, microalgae were resuspended in 850 μl of TAP at a concentration of 1.5·10⁸ cells/ml, and mixed, in a 1:1 ratio, with human fibrinogen (EVICEL®, Johnson & Johnson). Next, 850 μl of Thrombin (EVICEL®, Johnson Johnson) were homogeneously added to the scaffolds dropwise, followed by the addition of the 1700 μl of microalgae-fibrinogen solution. Matrices were left undisturbed for 1 h to ensure complete polymerization. Afterwards, the photosynthetic scaffolds were covered with 20 ml of TAP, and a biopsy sample with 1 ml of TAP were taken for quality control, including microbiology testing of aerobic and anaerobic bacteria, fungi, and rapidly growing mycobacteria culture for four days. Briefly, the biopsy samples were homogenized in 500 μl of TAP under sterile conditions. For aerobic culture, 50 μl of the mixture were seeded on 10 cm Trypcase Soy agar 5% sheep blood (bioMerieux), chocolate agar PolyViteX VCAT3 (bioMerieux) and MacConkey agar plates (Becton Dickinson). Additionally, 50 μl were also inoculated in Brain-Heart infusion broth (Becton Dickinson). For anaerobic culture, 50 μl of the homogenized mixture were seeded on Schaedler agar (Becton Dickinson), and inoculated in Thioglycollate broth. For fungi culture, 50 μl of homogenized mixture were seeded on Sabouraud agar plates. All plates and tubes were incubated at 35° C. for four days. Mycobacteria testing was performed by inoculating 50 μl of the mixture in 7 ml BD BACTEC™ MGIT™ mycobacteria growth indicator tubes, and protocol proceeded following manufacturer's instructions. In the case of any bacterial, fungi or mycobacterial detection, species were identified using a MALDI-TOF Microflex LT/SH mass spectrometer (Bruker Daltonik). During microbiology testing, scaffolds were left undisturbed at room temperature and with constant illumination (vide supra). Once safety was ensured by negative results from microbiology testing, photosynthetic scaffolds were sterilely packaged and transported to the operation room.

An illumination device was created and used for the controlled illumination of implanted photosynthetic scaffolds, composed of a control unit and a lighting system (Andes Scientific Instruments, Sky-Walkers SpA, Talagante, Chile). All suitable illumination devices and systems as described herein are contemplated. Electronics of the lighting system can be based on Pulse Width Modulation (PWM) intensity control of blue LEDs (wavelength 455 nm) or any other suitable LEDs or lighting elements through an electronic driver. LEDs can be soldered on a printed circuit board (PCB) and connected through flat ribbon cables, all supported by a copper sheet with a living hinge pattern. The electronics and batteries (e.g., rechargeable lithium-ion) can be held in a portable control unit with standard fixing to the institutional clinical holders. A user interface can allow programming of the LEDs intensity from 0 to 100% (e.g., from a maximal 550 mW per LED) and light schedule, e.g., light/dark 8/16 hours, via, for example, a Bluetooth™ application for remote control of the device. An LCD screen of the control device (or a separate device) can continuously display the battery state and temperature of the electronic driver, the copper sheet and the batteries.

The correct performance of the illumination device was validated by measuring the metabolic activity of C. reinhardtii, using an Oxygraph+ System with a Clark type electrode (Hansatech Instruments). Microalgae were resuspended in TAP at a concentration of 107 cells/ml, and 2 ml of the solution were pipetted into the electrode chamber. The illumination device was positioned 3 cm away from the chamber, and illuminated in cycles of 5/10 minutes of darkness/light, while variations in dissolved oxygen concentrations were recorded along the experimental time (FIG. 8).

Scaffold Implantation and Patient Follow Up

Before photosynthetic scaffold implantation, microbiology testing of the wound bed was performed once homogeneous granulation occurred, to ensure wound sterility by obtaining a negative tissue culture result. However, for patients scheduled for resection of a contracture scar, no previous microbiology testing was performed, since at the time of resecting the scar, the healthy underlying tissue is exposed.

All procedures were performed in the operating room at Hospital del Salvador (Santiago, Chile), under general or spinal anesthesia and strict aseptic technique. First, surgical debridement of the wound or scar removal was performed. Photosynthetic scaffolds were implanted onto the defect area, fixed with non-absorbable monofilament nylon 4/0 sutures (Ethilon®, Johnson & Johnson) to the wound edges. Tegaderm® (3M) dressing was positioned over the scaffolds, followed by a thin sterile gauze. Finally, an illumination device was positioned on top of the dressings, secured with an elastic bandage. The light control unit was turned on and LED intensity was configured by Bluetooth via Android application at the end of surgical time. Patients were hospitalized for seven days for daily follow-up of the implanted photosynthetic scaffold. After day 7, the illumination system was withdrawn, and hospital discharge was granted, continuing with outpatient control.

A second surgical procedure was performed 21 days after scaffold implantation, once adequate adherence of the scaffold to the wound bed was achieved. Silicone sheet from the Integra® matrix was removed, and an autologous partial skin graft was obtained from the patient's thigh with the use of a dermatome (Acculan® 3Ti, Aesculap). Small serial fenestrations were performed to the skin graft to avoid accumulation of exudate under the graft, and placed on top of the implanted photosynthetic scaffold, being fixed with a nonabsorbable nylon suture (Ethilon®, Johnson & Johnson) to the surrounding skin. The autograft was secured with Negative pressure wound therapy (NPWT) (Renasys®, Smith & Nephew), and patients were hospitalized for 6 days. Prior to hospital discharge, the NPWT system was removed and substituted with traditional advanced dressings. Patients were kept under close outpatient follow-up for the next 90 days.

The systemic immune response of the patients against the photosynthetic scaffold was evaluated by means of hematological and biochemical blood profiles, as well as the concentration of plasma cytokines and immune cells in peripheral blood, at days 0 (before photosynthetic scaffold implantation), 3, 6, 9, 21 (before autografting), 24, 27, 36 and 90. Additionally, the local immune response was evaluated by taking biopsy samples on days 7 and 21 post scaffold implantation (before autografting), for further histological and immunohistochemical analysis.

A self-evaluation questionnaire was delivered to each patient throughout the first 10 days, where pain intensity, burning, itching, smell, and light annoyance were recorded using visual analog scale (VAS) or Likert proportional scale.

Clinical Laboratory Test

Hematological profiles, coagulations tests and biochemical profiles from whole blood samples were performed on all patients at the specific time points indicated above. Hematological profile included hematocrit as well as erythrocytes, hemoglobin, platelets and leukocytes counts by certified clinical laboratory methods. Coagulation tests included international normalized ratio (INR), prothrombin time (PT) and partial thromboplastin time (PTT). Additionally, biochemical profiles included quantification of blood glucose, creatinine, bilirubin direct and total levels, serum glutamic-oxaloacetic transaminase (SGOT), serum glutamic-pyruvic transaminase (SGPT), alkaline phosphatase and C-reactive protein, clinically relevant enzymatic activities and plasmatic electrolytes (sodium, potassium and chloride) performed by certified clinical laboratory techniques.

Immune Cell Populations

Lymphocyte subpopulations from peripheral blood samples were measured by flow cytometry. Cells were stained with the following monoclonal antibodies against surface markers: CD3/CD16&56 (BD Simultest, Cat. 340042), CD19 (PerCP-Cy5.5, BD, Cat. 340951), CD25 (PE-Cy7, BD Pharmingen, Cat. 557741), CD8 (APC, BD Pharmingen, Cat. 555369) and CD4 (APC-H7, BD Pharmingen, Cat. 560158). The analyses of lymphocyte subpopulations were performed on a FACSCanto II (BD Biosciences) cytometer. Data analysis was performed with FACSDiva™ clinical software (BD Biosciences).

Cytokine Analysis

Peripheral blood was collected from each subject in acid-citrate dextrose Vacutainers®. Plasma was obtained by blood centrifugation, which was further harvested, aliquoted and stored at −80° C. until cytokine analysis. Plasma concentrations of pro-inflammatory cytokines (IL-β, TNF-α, IL-6, IL-8 and IL-12p70), and anti-inflammatory cytokines (IL-10) were determined by a BD™ CBA (Cytokine Beads Array) Human Inflammation Kit (BD Biosciences), according to the protocol indicated by the manufacturer. Briefly, capture beads conjugated with a specific antibody and phycoerythrin (PE)-conjugated detector antibodies were incubated with samples. Specific fluorescent signals were measured by flow cytometry, compared to a calibration curve obtained with recombinant cytokines to obtain the bound analyte amount. The minimum detectable amount for the cytokines were as follows: IL-1β 2.4 pg/ml; TNF-α 4.0 pg/ml; IL-6 2.4 pg/ml; IL-8 1.7 pg/ml; IL-12p70 12.3 pg/ml and IL-10 3.7 pg/ml.

Histopathology and Immunohistochemistry

Biopsy samples were obtained on days 7 and 21 after photosynthetic scaffold implantation. The biopsies were fixed in a paraformaldehyde solution (4%), embedded in Paraplast (Leica Biosystems) at 60° C. Sections of 5 μm in thickness were cut and adhered to glass slides using 0.1% poly-L-Lysine (Sigma) and then dried at room temperature (25° C.). Prior to the immunoreaction, some samples were stained with hematoxylin and eosin (H-E) and Giemsa stain for morphological studies.

Immunohistochemistry was performed according to a previously established protocol. Sections were deparaffinized, rehydrated, and incubated with the primary anti-CD68 (Cat. MA5-13324, mouse monoclonal; ThermoFisher Scientific) 1:50, in PBS containing 0.3% (v/v) Tween 20, overnight at 4° C. Nonspecific staining was blocked by immersion in Cas-Block solution (ThermoFisher Scientific) and goat serum (Gibco) for 30 min (both). After extensive rinsing in PBS, all sections were incubated for 1 h at room temperature with biotin conjugated goat anti-rabbit IgG (Rockland Immunochemicals, Inc.) diluted 1:500 in PBS. The peroxidase reaction was visualized using the NovaRED kit (Vector Laboratories Inc.). After immunostaining, sections were lightly stained with Harris hematoxylin (Merck Millipore). For each immunohistochemical reaction, controls were performed by incubating the sections with PBS or by omitting the primary antibody. Histopathology control samples were performed, and are presented in the Supplementary Materials.

Sections were examined using a Leica DM500 microscope, and images were captured using a Leica ICC50 W digital camera integrated system and LAS EZ 3.4 software.

Statistical Analysis

Laboratory and values from patients were expressed as the arithmetic mean±standard deviation. Multivariate comparisons were evaluated by the non-parametric Kruskal-Wallis test with Bonferroni correction and post hoc Mann-Whitney test. The significance level was set at p<0.05.

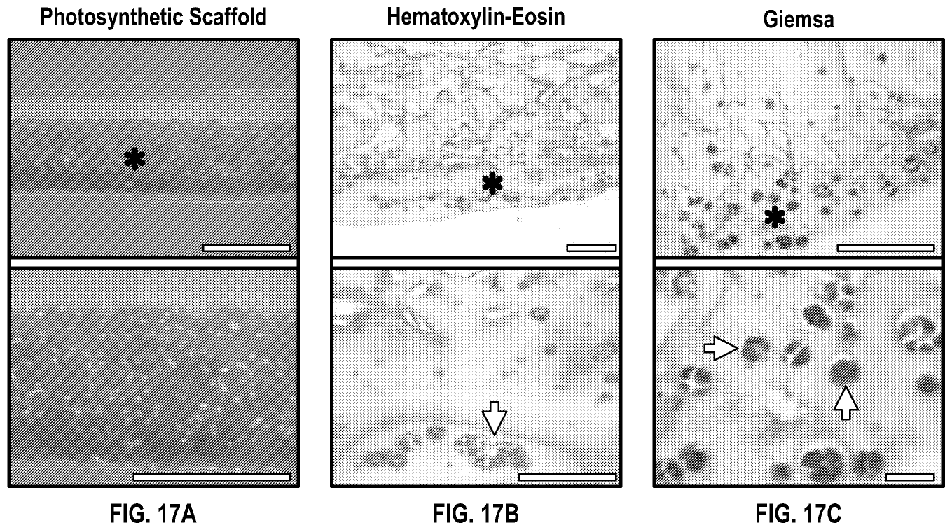

FIGS. 17A-17C illustrate histology of photosynthetic control scaffolds. FIG. 17A shows a photosynthetic scaffold cross section, showing the porous matrix covered with a silicon layer. In FIG. 17B, Hematoxylin-eosin stain of the scaffold shows the presence of collagen fibers and microalgae (arrow). In FIG. 17C, Microalgae (arrows) embedded in the scaffold are shown by Giemsa stain. Scale bars represent 2 mm in FIG. 17A, 200 μm in FIG. 17B (upper), 100 μm in FIG. 17B (lower) and FIG. 17C (upper), and 20 μm in FIG. 17C (lower). Asterisks in upper images show magnified area shown in the lower images.

Thus, specific examples of illumination devices and methods of using illumination devices have been disclosed. It should be apparent, however, to those skilled in the art that many more modifications besides those already described are possible without departing from the inventive concepts herein. While examples and variations of the many aspects of the invention have been disclosed and described herein, such disclosure is provided for purposes of explanation and illustration only. Thus, various changes and modifications may be made without departing from the scope of the claims.

Reference throughout this specification to “an embodiment” or “an implementation” means that a particular feature, structure, or characteristic described in connection with the embodiment is included in at least one embodiment or implementation. Thus, appearances of the phrases “in an embodiment” in various places throughout this specification are not necessarily all referring to the same embodiment or a single exclusive embodiment. Furthermore, the particular features, structures, or characteristics described herein may be combined in any suitable manner in one or more embodiments or one or more implementations.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise.

Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C.

All structural and functional equivalents to the components of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An illumination system, comprising:
an illumination device, comprising a metal substrate, a living hinge, and a set of lighting elements coupled to the metal substrate.

2. The illumination system of claim 1, wherein the metal substrate comprises copper.

3. The illumination system of claim 1, wherein the living hinge comprises cutouts on the metal substrate.

4. The illumination system of claim 1, wherein the set of lighting elements comprises light emitting diodes that are supported by a set of printed circuit boards and connected via a set of cables.

5. The illumination system of claim 1, wherein the set of lighting elements comprises optical fibers.

6. The illumination system of claim 1, further comprising a photosynthetic scaffold, and wherein the illumination device is wearable on a portion of a wearer having the photosynthetic scaffold, and wherein the illumination device comprises a fastener.

7. The illumination system of claim 1, further comprising a sensor attached or otherwise coupled to the metal substrate.

8. The illumination system of claim 1, further comprising a control unit coupled to the illumination device, and comprising a user interface.

9. The illumination system of claim 8, wherein the control unit is configured to adjust an intensity of the set of lighting elements via pulse-width modulation.

10. The illumination system of claim 1, wherein the set of lighting elements are arranged as rhomboids.

11. The illumination system of claim 1, wherein the set of lighting elements are arranged as hexagons.

12. The illumination system of claim 1, wherein the set of lighting elements comprise a set of optical fibers coupled to a set of LEDs.

13. The illumination system of claim 1, further comprising a control unit coupled to the wearable illumination device, and a sensor configured to collect data relating to at least one of the control unit and a user of the control unit, wherein the control unit is configured to adjust an intensity of the set of lighting elements based on data obtained by the sensor.

14. An illumination system, comprising:
an illumination device, comprising a metal substrate, and a set of lighting elements coupled to the metal substrate on a first side.

15. The illumination system of claim 14, wherein the metal substrate comprises copper.

16. The illumination system of claim 14, wherein the metal substrate comprises a living hinge.

17. The illumination system of claim 14, wherein the set of lighting elements comprises light emitting diodes that are supported by a set of printed circuit boards and connected via a set of cables.

18. The illumination system of claim 14, wherein the set of lighting elements comprises optical fibers.

19. The illumination system of claim 14, further comprising a sensor coupled to a control unit configured to adjust an intensity of the set of lighting elements based on sensor data obtained from the sensor.

20. The illumination system of claim 14, wherein the illumination device is an implantable device.

21. The illumination system of claim 14, wherein the illumination device is formed as a container and delivers light to an object housed therein.

22. A system, comprising:
a wearable illumination device, comprising:
a substrate;
a living hinge; and
a set of lighting elements coupled to the substrate; and
wherein the wearable illumination device is sized and dimensioned to be worn over a portion of a body of a wearer that comprises a skin defect with a photosynthetic scaffold placed thereon.
